(19) United States
(12) Patent Application Publication
(10) Pub. No.: US 2023/0139394 A1
(43) Pub. Date: May 4, 2023

(54) EEG BASED SPEECH PROSTHETIC FOR STROKE SURVIVORS

(71) Applicant: Austin Speech Labs, LLC, Austin, TX (US)

(72) Inventors: Gautam Krishna, Austin, TX (US); Mason Carnahan, Austin, TX (US); Amrit Chandar, Austin, TX (US); Shilpa Shamapant, Austin, TX (US); Ahmed Tewfik, Austin, TX (US); José del R. Millán, Austin, TX (US)

(73) Assignees: Austin Speech Labs, LLC, Austin, TX (US); Board of Regents, the University of Texas System, Austin, TX (US)

(21) Appl. No.: 17/647,616

(22) Filed: Jan. 11, 2022

Related U.S. Application Data
(60) Provisional application No. 63/273,079, filed on Oct. 28, 2021.

(51) Int. Cl. G10L 15/24; G10L 25/24; G10L 15/02; G10L 15/16; G10L 15/22; A61B 5/369; A61B 5/389; A61B 5/00; A61B 5/372

(52) U.S. Cl. CPC G10L 15/24; G10L 25/24; G10L 15/02; G10L 15/16; G10L 15/22; A61B 5/369; A61B 5/389; A61B 5/6815; A61B 5/372

(57) ABSTRACT

A method of electroencephalography (EEG) based speech recognition includes obtaining, from a microphone, an audio signal of a speaker from a first time period, obtaining, from one or more EEG sensors, EEG signals of the speaker from the first time period, obtaining, from a first model, acoustic representations based on the EEG signals, concatenating the obtained acoustic representations with an audio input based on the audio signal to obtain concatenated features, providing the concatenated features to an automatic speech recognition model (ASR) and obtaining, from the ASR model, a text-based output.

### REFERENCE TO RELATED APPLICATIONS

The present application is related to U.S. Provisional Patent Application No. 63/273,079 filed Oct. 28, 2021, the contents of which are incorporated by reference as if fully set forth herein.

### TECHNICAL FIELD

This disclosure relates to speech therapy and apparatus for decoding translating speech by persons with speech disorders, including, without limitation, aphasia, apraxia and dysarthria. Specifically, the present disclosure is directed to embodiments of an electroencephalography (EEG) based speech prosthetic for stroke survivors, and methods for operating same.

### BACKGROUND

For patients with certain speech conditions, notably Aphasia (dysfunction in the regions of the brain responsible for comprehension and formulation of language), Apraxia (impairment of speech-related motor planning) and Dysarthria (damage to the motor component of the motor-speech system), communication and accessibility are a persistent source of challenges. Beyond the social and personal challenges associated with the broken and/or distorted speech these conditions cause, these conditions' effects on patients' speech are such that by itself, many patients' speech cannot serve as a set of training data or features to be provided to sound-only automatic speech recognition models. As such, development of speech prosthetics to decode and translate such patients' speech has stalled due to the deficiencies of audio-only speech recognition models.

Additionally, for many patients, the initial trauma (for example, stroke) creating the speech conditions can be a source of clinical fragility, weighing against performing surgery to implant sensors.

Accordingly, developing non-invasive speech prosthetics for patients with speech conditions that preclude the application of audio-only speech recognition presents a significant source of technical challenges and opportunities for improvement in the art.

### SUMMARY

This disclosure provides examples of EEG based speech prosthetics for stroke survivors and methods for providing same.

Other technical features may be readily apparent to one skilled in the art from the following figures, descriptions, and claims.

In a first embodiment, a method of electroencephalography (EEG) based speech recognition includes obtaining, from a microphone, an audio signal of a speaker from a first time period, obtaining, from one or more EEG sensors, EEG signals of the speaker from the first time period, obtaining, from a first model, acoustic representations based on the EEG signals, concatenating the obtained acoustic representations with an audio input based on the audio signal to obtain concatenated features, providing the concatenated features to an automatic speech recognition model (ASR) and obtaining, from the ASR model, a text-based output.

In a second embodiment, an apparatus for performing electroencephalography (EEG) based speech recognition includes an input/output interface and a processor configured to obtain, from a microphone, via the input/output interface, an audio signal of a speaker from a first time period, obtain, from one or more EEG sensors, via the input/output interface, EEG signals of the speaker from the first time period, obtain, from a first model, acoustic representations based on the EEG signals, concatenate the obtained acoustic representations with an audio input based on the audio signal to obtain concatenated features, provide the concatenated features to an automatic speech recognition model (ASR), and obtain, from the ASR model, a text-based output.

Before undertaking the DETAILED DESCRIPTION below, it may be advantageous to set forth definitions of certain words and phrases used throughout this patent document. The term “couple” and its derivatives refer to any direct or indirect communication between two or more elements, whether or not those elements are in physical contact with one another. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like. The term “controller” means any device, system or part thereof that controls at least one operation. Such a controller may be implemented in hardware or a combination of hardware and software and/or firmware. The functionality associated with any particular controller may be centralized or distributed, whether locally or remotely. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

Definitions for other certain words and phrases are provided throughout this patent document. Those of ordinary skill in the art should understand that in many if not most instances, such definitions apply to prior as well as future uses of such defined words and phrases.

### BRIEF DESCRIPTION OF THE DRAWINGS

For a more complete understanding of this disclosure and its advantages, reference is now made to the following description, taken in conjunction with the accompanying drawings, in which:

FIG. 1 illustrates a non-limiting example of an electronic device according to some embodiments of this disclosure;

FIG. 2 illustrates an example of a server according to certain embodiments of this disclosure;

FIG. 3 illustrates an architecture for training an automatic speech recognition (ASR) model utilizing EEG-based features according to various embodiments of this disclosure;

FIG. 4 illustrates an example of an isolated speech recognition model according to certain embodiments of this disclosure;

FIG. 5 illustrates an example of a continuous speech recognition model, according to various embodiments of this disclosure;

FIG. 6 illustrates an example of a speaker ID recognition model, according to some embodiments of this disclosure;

FIG. 7 illustrates an example of a voice activity detection model, according to various embodiments of this disclosure;

FIG. 8 illustrates an example of a speech recognition decoding pipeline, according to some embodiments of this disclosure; and

FIG. 9 illustrates operations of an example method for performing EEG-based automatic speech recognition, according to certain embodiments of this disclosure.

### DETAILED DESCRIPTION

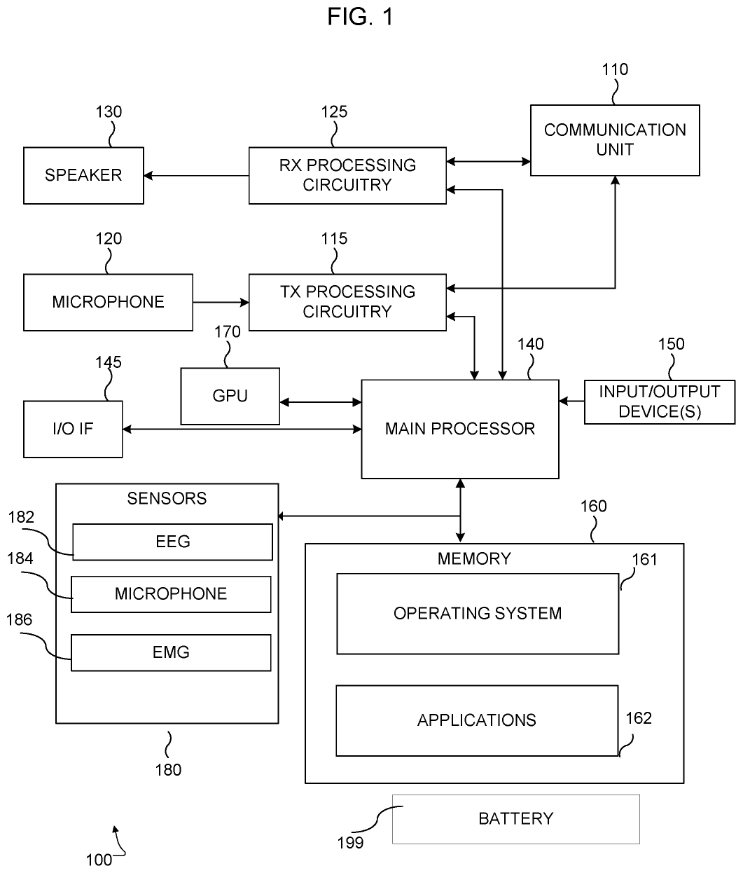

FIGS. 1 through 9, discussed below, and the various embodiments used to describe the principles of this disclosure in this patent document are by way of illustration only and should not be construed in any way to limit the scope of the disclosure. Those skilled in the art will understand that the principles of this disclosure may be implemented in any suitably arranged processing platform.

FIG. 1 illustrates a non-limiting example of a device 100 which can be configured to operate as an EEG based speech prosthetic according to some embodiments of this disclosure. According to various embodiments of this disclosure, device 100 could be implemented as one or more of a smartphone, a tablet, a laptop computer, a digital home assistant (for example, an AMAZON ALEXA® type device), an interactive voice response (IVR) system, a voice-controlled appliance, a smart speaker, a virtual assistant, a wearable device or other processor based apparatus. The embodiment of device 100 illustrated in FIG. 1 is for illustration only, and other configurations are possible. However, suitable devices come in a wide variety of configurations, and FIG. 1 does not limit the scope of this disclosure to any particular implementation of a device.

As shown in the non-limiting example of FIG. 1, the device 100 includes a communication unit 110 that may include, for example, a radio frequency (RF) transceiver, a BLUETOOTH transceiver, or a WI-FI transceiver, etc., transmit (TX) processing circuitry 115, a microphone 120, and receive (RX) processing circuitry 125. The device 100 also includes a speaker 130, a main processor 140, an input/output (I/O) interface (IF) 145, input/output device(s) 150, and a memory 160. The memory 160 includes an operating system (OS) program 161 and one or more applications 162.

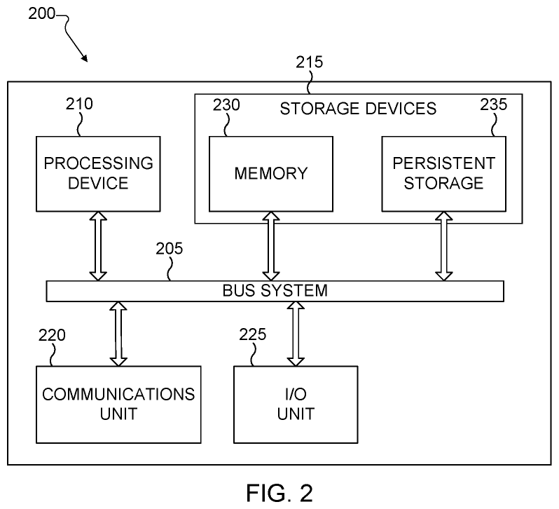

Applications 162 can include web browsers, health monitoring applications, applications for maintaining a client-host relationship between device 100 and a host device (for example, server 200 in FIG. 2) operating systems, device security (e.g., anti-theft and device tracking) applications or any other applications providing or supporting an EEG-assisted speech recognition functionality. According to some embodiments, the resources of device 100 include, without limitation, speaker 130, microphone 120, input/output devices 150, and sensors 180.

Referring to the non-limiting example of FIG. 1, sensors 180 comprise one or more external electroencephalography (EEG) sensors 182, which are configured to measure electrical impulses associated with brain activity through the skin. In some embodiments, EEG sensor(s) 182 comprise wet EEG sensors, typically provided as an EEG cap, wherein electrode sensors are held in substantially fixed contact with predetermined points on a user's scalp via a swim-cap like structure, and a layer of a wet, conductivity-enhancing medium (for example, water or a saline gel) is provided between each electrode and the wearer's scalp. In various embodiments, the predetermined points correspond to points on the scalp closest to the parts of the temporal and frontal lobes containing the brain regions responsible for speech perception and production. For example, the electrodes of EEG sensor(s) 182 may be positioned at the points designated Fp1, Fz, F3, F7, FT9, FC5, FT10, FC6, FC 2, F4, F8, Fp2, T7, TP9 and T8 according to the 10-20 EEG sensor placement guidelines. Depending on embodiments, EEG sensor(s) may be placed at more or fewer locations. According to certain embodiments EEG sensor(s) 182 may alternatively, or additionally comprise one or more dry EEG sensors (for example, Brain Products' ActiCAP Xpress electrodes), which can be affixed directly to a patient's scalp without an EEG cap or the use of a wet conductive medium. For certain patients and clinical settings, dry EEG sensors may be preferable to wet EEG sensors. Additionally, in some embodiments, EEG sensor(s) 182 comprise an array of sensors (for example, 9 sensors per ear) disposed around the periphery of each of the patient's ears.

In some embodiments, signals from EEG sensor(s) 182 may be amplified (for example, using BRAIN PRODUCTS' ACTICHAMP AMPLIFIER) prior to being provided to main processor 140. According to some embodiments, samples from EEG sensor(s) 182 are obtained at a predetermined sampling frequency (for example, 1000 Hz) and filtered to remove ambient noise. In some embodiments, signals from EEG sensor(s) 182 may be passed through a bandpass filter (for example, a Butterworth filter) and then a notch filter with a cut off frequency of 60 Hz (to remove power line noise).

Referring to the illustrative example of FIG. 1, sensors 180 may further comprise one or more microphones 184. According to some embodiments, microphone 184 is a directional microphone either integrated in, or connected to device 100.

As shown in the explanatory example of FIG. 1, sensors 180 can include one or more electromyography (EMG) sensors 186 to be mounted on a patient's head or face to pick up electrical impulses from facial muscles, which can confound or add noise to brain signals measured by EEG sensor(s) 182.

The communication unit 110 may receive an incoming RF signal, for example, a near field communication signal such as a BLUETOOTH or WI-FI signal. The communication unit 110 can down-convert the incoming RF signal to generate an intermediate frequency (IF) or baseband signal. The IF or baseband signal is sent to the RX processing circuitry 125, which generates a processed baseband signal by filtering, decoding, or digitizing the baseband or IF signal. The RX processing circuitry 125 transmits the processed baseband signal to the speaker 130 (such as for voice data) or to the main processor 140 for further processing. Additionally, communication unit 110 may contain a network interface, such as a network card, or a network interface implemented through software. In this way, device 100 can receive data (for example, updates to speech recognition models or models for processing and extracting features from EEG data).

The TX processing circuitry 115 receives analog or digital voice data from the microphone 120 or other outgoing baseband data from the main processor 140. The TX processing circuitry 115 encodes, multiplexes, or digitizes the outgoing baseband data to generate a processed baseband or IF signal. The communication unit 110 receives the outgoing processed baseband or IF signal from the TX processing circuitry 115 and up-converts the baseband or IF signal to an RF signal for transmission.

The main processor 140 can include one or more processors or other processing devices and execute the OS program 161 stored in the memory 160 in order to control the overall operation of the device 100. For example, the main processor 140 could control the reception of forward channel signals and the transmission of reverse channel signals by the communication unit 110, the RX processing circuitry 125, and the TX processing circuitry 115 in accordance with well-known principles. In some embodiments, the main processor 140 includes at least one microprocessor or microcontroller. According to certain embodiments, main processor 140 is a low-power processor, such as a processor which includes control logic for minimizing consumption of battery 199 or minimizing heat buildup in device 100.

The main processor 140 is also capable of executing other processes and programs resident in the memory 160. The main processor 140 can move data into or out of the memory 160 as required by an executing process. In some embodiments, the main processor 140 is configured to execute the applications 162 based on the OS program 161 or in response to inputs from a user or applications 162. Applications 162 can include applications specifically developed for the platform of device 100, or legacy applications developed for earlier platforms. The main processor 140 is also coupled to the I/O interface 145, which provides the device 100 with the ability to connect to other devices such as laptop computers and handheld computers. The I/O interface 145 is the communication path between these accessories and the main processor 140.

The main processor 140 is also coupled to the input/output device(s) 150. The operator of the device 100 can use the input/output device(s) 150 to enter data into the device 100. Input/output device(s) 150 can include keyboards, touch screens, mouse(s), track balls or other devices capable of acting as a user interface to allow a user to interact with device 100. In some embodiments, input/output device(s) 150 can include a touch panel, an augmented or virtual reality headset, a (digital) pen sensor, a key, or an ultrasonic input device.

Input/output device(s) 150 can include one or more screens, which can be a liquid crystal display, light-emitting diode (LED) display, an optical LED (OLED), an active-matrix OLED (AMOLED), or other screens capable of rendering graphics.

The memory 160 is coupled to the main processor 140. According to certain embodiments, part of the memory 160 includes a random-access memory (RAM), and another part of the memory 160 includes a Flash memory or other read-only memory (ROM). Although FIG. 1 illustrates one example of a device 100. Various changes can be made to FIG. 1.

For example, according to certain embodiments, device 100 can further include a separate graphics processing unit (GPU) 170.

According to various embodiments, the above-described components of device 100 are powered by a power source, and in one embodiment, by a battery 199 (for example, a rechargeable lithium-ion battery), whose size, charge capacity and load capacity are, in some embodiments, constrained by the form factor and user demands of the device. As a non-limiting example, in embodiments where device 100 is a smartphone or portable device (for example, a device worn by a patient), battery 199 is configured to fit within the housing of the device and is configured not to support current loads (for example, by running a graphics processing unit at full power for sustained periods) causing heat buildup.

Although FIG. 1 illustrates one example of a device 100 for providing a collaborative user interface, various changes may be made to FIG. 1. For example, the device 100 could include any number of components in any suitable arrangement. As one illustrative example, device 100 could be embedded in a larger system. In general, devices including computing and systems control platforms come in a wide variety of configurations, and FIG. 1 does not limit the scope of this disclosure to any particular configuration. While FIG. 1 illustrates one operating environment in which various features disclosed in this patent document can be used, these features could be used in any other suitable system.

FIG. 2 illustrates an example of a server or computer system 200 that can be used to perform EEG-based speech recognition operations according to certain embodiments of this disclosure, or as a high-powered computing platform for developing and training models to be loaded onto other devices (for example, device 100 in FIG. 1). The embodiment of the server 200 shown in FIG. 2 is for illustration only and other embodiments could be used without departing from the scope of the present disclosure. According to certain embodiments, the server 200 operates as a gateway for data passing between a device of a secure internal network (for example, device 100 in FIG. 1), and an unregulated external network, such as the internet.

In the example shown in FIG. 2, the server 200 includes a bus system 205, which supports communication between at least one processing device 210, at least one storage device 215, at least one communications unit 220, and at least one input/output (I/O) unit 225.

The processing device 210 executes instructions that may be loaded into a memory 230. The processing device 210 may include any suitable number(s) and type(s) of processors or other devices in any suitable arrangement. Example types of processing devices 210 include microprocessors, microcontrollers, digital signal processors, field programmable gate arrays, application specific integrated circuits, and discrete circuitry. In certain embodiments, the server 200 can be part of a cloud computing network, and processing device 210 can be an instance of a virtual machine or processing container (for example, a MICROSOFT AZURE CONTAINER INSTANCE, or a GOOGLE KUBERNETES container).

The memory 230 and a persistent storage 235 are examples of storage devices 215, which represent any structure(s) capable of storing and facilitating retrieval of information (such as data, program code, and/or other suitable information on a temporary or permanent basis). The memory 230 may represent a random-access memory or any other suitable volatile or non-volatile storage device(s). The persistent storage 235 may contain one or more components or devices supporting longer-term storage of data, such as a ready only memory, hard drive, Flash memory, or optical disc. According to various embodiments, persistent storage 235 is provided through one or more cloud storage systems (for example, AMAZON S3 storage).

The communications unit 220 supports communications with other systems or devices. For example, the communications unit 220 could include a network interface card or a wireless transceiver facilitating communications over the network 102. According to some embodiments, sensors for collecting speech-related data from a patient (for example, sensors 180 in FIG. 1) may connect directly to communications unit 220. Alternatively, or additionally, in some embodiments, speech-related EEG, EMG and audio data may be obtained on separately platforms (for example, device 100 in FIG. 1) and provided to server 200 via communications unit 220. The communications unit 220 may support communications through any suitable physical or wireless communication link(s).

The I/O unit 225 allows for input and output of data. For example, the I/O unit 225 may provide a connection for user input through a keyboard, mouse, keypad, touchscreen, or other suitable input device. The I/O unit 225 may also send output to a display, printer, or other suitable output device.

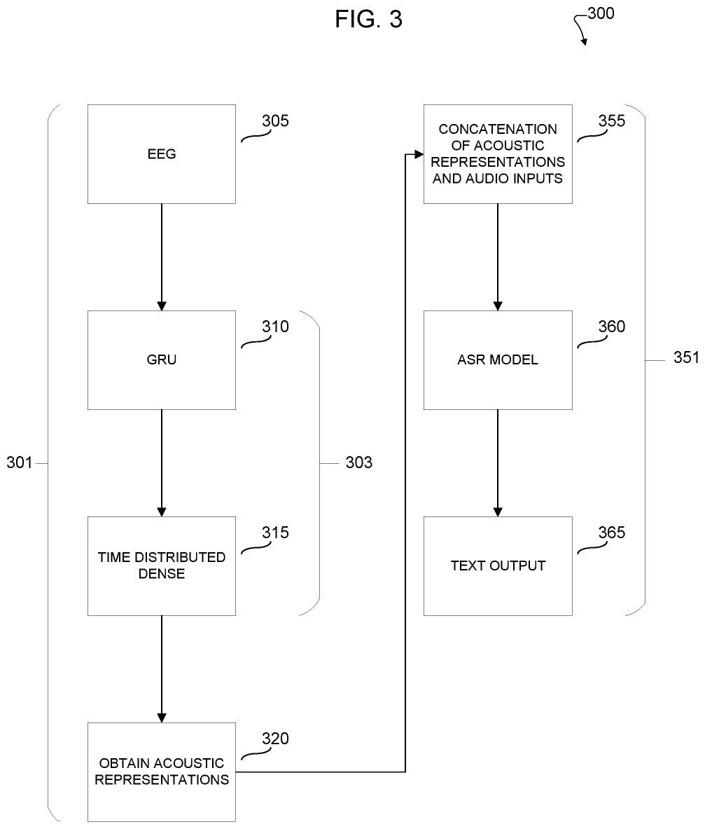

FIG. 3 illustrates an architecture 300 for training an automatic speech recognition (ASR) model 360 utilizing EEG-based features, according to various embodiments of this disclosure. In this way, certain embodiments according to this disclosure can, if desired, leverage the existing training and development of certain ASR models by providing additional EEG-augmented features to assist the training of ASR model 360 to better decode and recognize speech inputs from patients with conditions degrading the performance of speech-related areas of the brain. Put differently, certain embodiments according to this disclosure provide EEG enriched features to ASR model 360 such that ASR model 360 can be trained to be more performant in recognizing speech from patients with aphasia, apraxia, dysarthria or other conditions degrading or distorting the patients' speech.

Referring to the explanatory of FIG. 3, architecture 300 is a two-stage architecture comprising a deep learning regression stage 301 and a speech recognition stage 351. In this example, regression stage 301 is initially trained to predict acoustic representations (for example, Mel frequency cepstral coefficients (MFCCs)) from EEG data. Once regression stage 301 is sufficiently trained, the predicted acoustic representations output by regression stage 301 are concatenated with an audio input based on data obtained over the same time interval as the EEG data to train ASR model 360, which in turn, outputs recognized text-based outputs.

FIG. 3 provides a non-limiting example of an architecture in which audio signals obtained from a speaker can be enriched with contemporaneous EEG signals obtained from the speaker to provide an underlying data set that is sufficiently feature-rich to reliably recognize the speech from individuals with conditions that impair or distort their speech beyond what audio-only speech recognition models can recognize. As such, architecture 300 may be implemented across a wide range of contexts to support a wide variety of practical applications. For example, in some embodiments, architecture 300 can be used, in conjunction with language laboratory apparatus to enhance speech therapy and shorten the recovery time for patients with aphasia, apraxia and dysarthria, by improving sentence recognition and reinforcement of patients' recovery of disrupted speech skills. Additionally, architecture 300 may, in some embodiments, be implemented on a portable computing platform, and can operate as a translator of patients' speech, enhancing their ability to operate independently and communicate with others.

As shown in FIG. 3, at block 305, EEG signals from a speaker are obtained from one or more EEG sensors (for example, EEG sensor(s) 182 in FIG. 1) during a specified time interval. While not processed as part of deep learning regression stage 301, audio signals of the speaker (for example, audio captured by microphone 184 in FIG. 1) during the specified time period are also obtained.

According to various embodiments, the EEG signals may be obtained from wet EEG sensors, dry EEG sensors, EEG sensors located around a user's ear, or combinations thereof. Further, in some embodiments, the EEG signals may be pre-processed by being passed through one or more filters. For example, in some embodiments, where the recorded EEG signals were sampled at a sampling frequency of 1000 Hertz (Hz), the EEG signals were passed through a fourth-order infinite impulse response (IIR) bandpass filter with cut off frequencies of 0.1 Hz and 70 Hz. Further, in certain embodiments, the EEG signals were passed through a notch filter with a cutoff frequency of 60 Hz to remove power line noise.

Referring to the non-limiting example of FIG. 3, in some embodiments, in addition to audio and EEG signals, EMG signals are obtained (for example, from EMG sensor 186 in FIG. 1) during the specified time interval from sensors on the speaker's skin (for example, along the speaker's jawline and lower chin). In this way, at block 305, EMG artifacts can be removed from the EEG signals using the linear regression represented by Equation 1, below:

Corrected_{EEG}=Recorded_{EEG}−α*Recorded_{EMG} (1)

Where α is the regression coefficient computed by an ordinary least squares method.

Further, at block 305, feature extraction is performed on the filtered EEG signals. In this example, the output of each EEG sensor contacting the speaker during the time interval comprises a channel of the EEG data. For each channel of the filtered EEG signals, the following features may be extracted from each channel's data: root mean square values over specified sub-intervals, a quantification of the spectral entropy of the channel's data, values of a moving average of the values over specified sub-intervals, a zero-crossing rate within each channel's data, and a quantification of the presence of outliers (i.e., kurtosis) in the distribution of values within each channel's data. In some embodiments, the aforementioned EEG features are determined at a rate approximately equal to one tenth of the rate at which the EEG data is sampled. Thus, if data from a given sensor on a speaker's scalp is sampled from a given EEG sensor is sampled at 1000 Hz, features in the data may be sampled at a rate of 100 Hz.

Referring to the non-limiting example of FIG. 3, the extracted EEG features are provided to a deep learning model 303 which can be trained, through unsupervised learning, to recognize patterns (referred to herein as “acoustic representations”) within the extracted EEG features corresponding to features in audio signals provided to automatic speech recognition model 360. In certain embodiments, the acoustic representations are Mel frequency cepstral coefficients (MFCC) having a predetermined dimensionality (for example, 13).

According to various embodiments, deep learning model 303 comprises, as a first layer, a gated recurrent unit (GRU) 310 with a predetermined (for example, 128) number of hidden units, which is connected to a time distributed dense layer 315 with a number of hidden units corresponding to the dimensionality of the acoustic representations output by deep learning model 303. Using a training set of approximately 5,000 data samples, wherein each data sample included 29 channels of EEG data, deep learning model 303 was trained by passing through GRU 310 and time distributed dense layer 315 for 70 epochs with mean square error (MSE) as the loss function and adaptive moment estimation (“Adam”) as the optimizer. In some embodiments, other loss functions and optimizers (for example, ADADELTA) may be used, and the scope of the present disclosure should not be construed as being limited to any one specific combination of loss function and optimizer.

According to various embodiments, upon completion of training deep learning model 303, EEG signals obtained over a common time interval as audio signals are passed through trained deep learning model 303 to obtain, at block 320, a set of acoustic representations (for example, MFCCs) based on the EEG signals.

Having trained deep learning regression model 303, training proceeds to second speech recognition stage 351, wherein an ASR model 360 is trained based, at least in part, on the acoustic representations obtained at block 320.

Referring to the illustrative example of FIG. 3, at block 355, an audio input based on audio signals obtained over the same time interval as the EEG inputs providing acoustic representations (for example, the acoustic representations obtained at 320) is obtained and concatenated with the acoustic representations. According to various embodiments, obtaining the audio input comprises passing the audio signals through a pre-processing pipeline to obtain a second set of Mel frequency cepstral coefficients based on the audio signals obtained during the common time interval. According to various embodiments, the audio signals may have a relatively high initial sampling rate (for example 16 KHz).

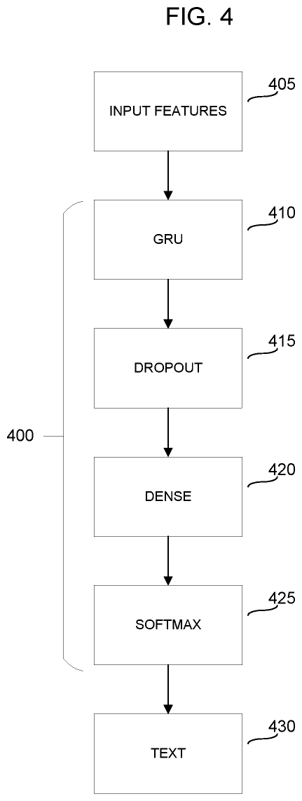

In some embodiments, the audio pre-processing pipeline comprises performing, a Fourier transform of the audio signals, followed by mapping the power values of the Fourier transform to the Mel scale. Subsequently, a log of the powers is taken and a discrete cosine transform is performed to obtain a representation of the amplitude of the constituent frequency spectrums of the audio signals, from which a second set of Mel frequency cepstral coefficients is obtained. According to various embodiments, the obtained Mel frequency cepstral coefficients are of the same dimensionality as those obtained at block 320. According to certain embodiments, the obtained Mel frequency cepstral coefficients are obtained at the same sampling rate as those obtained at block 320. According to various embodiments, at block 355, the obtained audio input is concatenated with the acoustic representations obtained at block 320 to form an enriched training set for training one or more ASR model(s) 360. The architecture and training of ASR model(s) 360 depends on the task to be performed and the recognized text-based outputs sought. According to various embodiments, ASR model(s) 360 may be one or more of an isolated speech recognition model, a continuous speech recognition model, a speaker identification model or a voice activity detection model. Non-limiting examples of the architectures for such models and training methods are provided in FIGS. 4-7 of this disclosure. Once ASR model(s) 360 are trained, architecture 300 can operate as a speech recognition pipeline, receiving simultaneously recorded EEG signal and audio signal data, and providing recognized text-based outputs 365. According to various embodiments, the recognized text-based outputs 365 may be reproduced through a text-to-speech application to improve their intelligibility. Alternatively, or additionally, recognized text-based outputs 365 may be passed as control inputs to another system, such as an internet of things (IoT) hub controlling the lights or thermostat of a room. In this way, embodiments according to the present disclosure allow patients with conditions distorting or otherwise degrading their speech, the same level of verbal control over IoT apparatus as individuals without such conditions.

FIG. 4 illustrates aspects of the architecture and training of an isolated speech recognition automatic speech recognition model (ASR) 400 according to various embodiments of this disclosure. According to certain embodiments, ASR model 400 may be implemented on any suitable processing platform, including, without limitation, device 100 in FIG. 1 or server 200 in FIG. 2. Additionally, ASR 400 may be part (for example, ASR 360 in FIG. 3) of a multi-stage architecture for EEG based speech recognition.

As used in this disclosure, the expression “isolated speech recognition” encompasses sentence or verbal sequence classification tasks wherein an ASR model decodes a closed vocabulary and directly learns a mapping between input features and a sentence or other verbal structure associated with a label token. Put differently, ASR 400 provides, as a recognized text-based output, a prediction of a complete sentence or label token based on the obtained audio and EEG signals.

Referring to the non-limiting example of FIG. 4, for both training and prediction, the input features 405 of ASR model 400 are a concatenation (for example, the concatenation obtained at block 355 of FIG. 3) of acoustic representations obtained from EEG signals over a sample time and audio inputs obtained from audio signals over the sample time. Architecturally, ASR model 400 comprises a single layer gated recurrent unit (GRU) 410 with a predetermined number of hidden units. In some embodiments, GRU 410 comprises 512 hidden units, and in some embodiments, GRU 410 comprises 256 hidden units. However, skilled artisans will appreciate that other embodiments, with greater or fewer hidden units, are possible and within the contemplated scope of this disclosure.

According to various embodiments, ASR model 400 further comprises a dropout regularization function 415, which is configured to “drop out” or ignore a randomized fraction of the outputs of the nodes of GRU 410. In this way, the risk of overfitting ASR model 400 to its training data is mitigated. According to certain embodiments, dropout regularization function 415 is configured to have a drop-out rate of 0.2, which has been experimentally shown to strike a good balance between generalization and avoiding overfitting. However, other embodiments with different drop-out rates are possible and within the contemplated scope of this disclosure.

Following application of dropout regularization function 415, the outputs of GRU 410 are provided to a dense layer 420 comprising a number of hidden units corresponding to a number of sentences or label tokens in the output space. That is, in embodiments where, for example, ASR 400 has been trained to recognize 56 sentences from input features 405, dense layer 420 comprises 56 hidden units.

According to various embodiments, the output of dense layer 420 are provided to softmax activation function 425 to obtain a vector of prediction probabilities, wherein each prediction probability is associated with a single sentence or label token in the training set. According to various embodiments, ASR 400 could be trained on a training set comprising approximately 5000 data samples within 10 epochs and with a batch size of fifty. In certain embodiments, ASR 400 was trained using categorical cross-entropy as a loss function and Adam as an optimizer. To further mitigate over-fitting, early stopping was used during training. However, embodiments in which, for example, different loss and optimization functions are utilized are possible and within the contemplated scope of this disclosure.

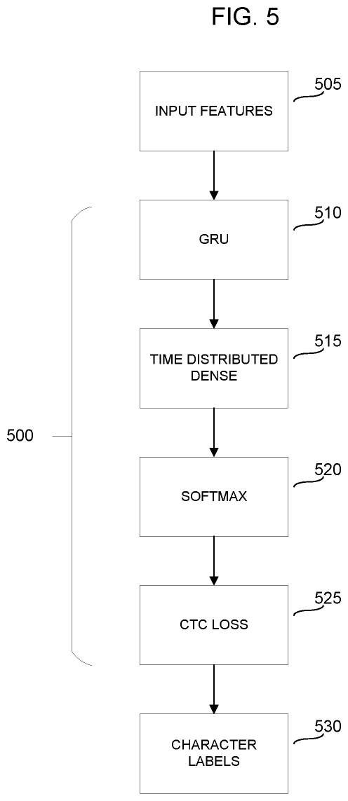

FIG. 5 illustrates aspects of the architecture and training of a continuous speech recognition automatic speech recognition model (ASR) 500 according to various embodiments of this disclosure. According to certain embodiments, ASR model 500 may be implemented on any suitable processing platform, including, without limitation, device 100 in FIG. 1 or server 200 in FIG. 2. Additionally, ASR 500 may be part (for example, ASR 360 in FIG. 3) of a multi-stage architecture for EEG based speech recognition.

As used in this disclosure, the expression “continuous speech recognition” refers to tasks in which an ASR model predicts the text of the speaker's speech by predicting the character, word or phoneme at every time step. As such, continuous speech recognition can provide greater opportunities for open vocabulary decoding, albeit at an increase in the complexity of the ASR model.

Referring to the non-limiting example of FIG. 5, for both training and prediction, the input features 505 of ASR model 500 are a concatenation (for example, the concatenation obtained at block 355 of FIG. 3) of acoustic representations obtained from EEG signals over a sample time and audio inputs obtained from audio signals over the sample time. As shown in the explanatory example of FIG. 5, input features 505 are provided to a GRU layer 510 operating as an encoder. The output of GRU layer 510 is provided to a dense layer 515 configured to implement a 4-gram language model, whose outputs are characters associated with the input features 505 provided at each time step (for example, a time step comprising 1/100^{th} of a second). As shown in FIG. 5, the output of dense layer 515 is provided to a softmax activation function 520, which, in turn provides an input to a connectionist temporal classification (CTC) loss function 525 configured to output labeled probabilities of the characters 530 provided in the input features 505 for a given time step. According to certain embodiments, ASR 500 may, using a training set comprising approximately 5000 data samples, be trained across 100 epochs, with a batch size of 50, using an Adam optimizer and CTC loss function 525. Other embodiments, in which ASR 500 utilizes, for example, a different optimizer or loss function, are possible and within the contemplated scope of this disclosure.

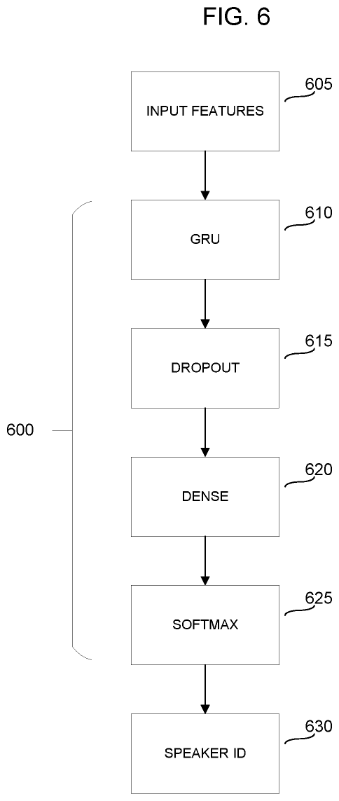

FIG. 6 illustrates aspects of the architecture and training of a speaker identification automatic speech recognition model (ASR) 600 according to various embodiments of this disclosure. According to certain embodiments, ASR model 600 may be implemented on any suitable processing platform, including, without limitation, device 100 in FIG. 1 or server 200 in FIG. 2. Additionally, ASR 600 may be part (for example, ASR 360 in FIG. 3) of a multi-stage architecture for EEG based speech recognition.

As used in this disclosure, “speaker ID recognition” encompasses generating based on input features 605, a vector of probabilities associating the input sounds and EEG signals with a labeled speaker in a training set. Put differently, ASR outputs a set of probabilities as to the source of a section of recorded speech. Referring to the non-limiting example of FIG. 6, for both training and prediction, the input features 605 of ASR model 600 are a concatenation (for example, the concatenation obtained at block 355 of FIG. 3) of acoustic representations obtained from EEG signals over a sample time and audio inputs obtained from audio signals over the sample time.

According to various embodiments, for both training and prediction, input features 605 are provided to a GRU 610 comprising a plurality of hidden features. In some embodiments, GRU 610 comprises 512 hidden features. In some embodiments, GRU 610 comprises 256 hidden features. Other embodiments, with fewer or greater hidden features, are possible, and within the contemplated scope of this disclosure. Referring to the non-limiting example of FIG. 6, GRU 610 is connected to a dropout regularization function 615, which, to mitigate the risk of overfitting, randomly removes a fraction of the outputs of GRU 610. According to various embodiments, dropout regularization function 615 implements a dropout regularization value of 0.2. However, embodiments with greater or less regularization are possible and within the contemplated scope of this disclosure.

Referring to the illustrative example of FIG. 6, following regularization by dropout regularization function 615, the outputs of the GRU are passed to a dense layer 620, wherein dense layer 620 comprises a number of hidden units corresponding to the number of speakers in the training data. For example, where ASR 600 is trained upon to recognize a speaker from a training set comprising ten speakers, dense layer 620 comprises 10 hidden units.

As shown in FIG. 6, the output of dense layer is provided to a softmax activation function 625, which provides an output 630 comprising a vector of prediction probabilities wherein each value of the vector is associated with candidate speaker the training set. In one embodiment, for a training set of nine speakers, model 600 was trained for ten (10) epochs with a batch size of 50. In certain embodiments, model 600 was trained using a categorical cross-entropy loss function and Adam as an optimizing function.

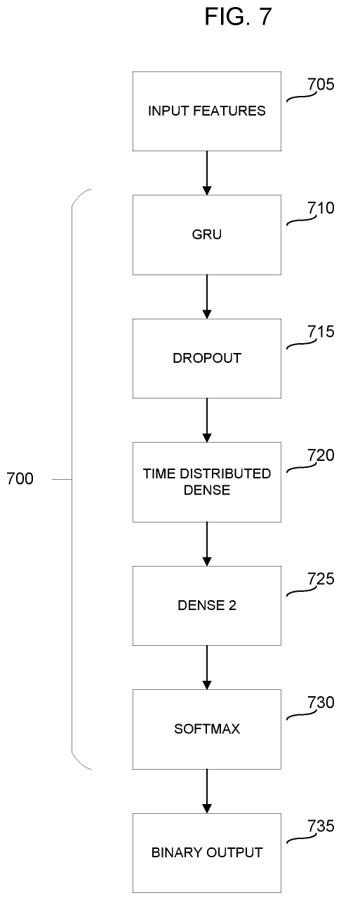

FIG. 7 illustrates aspects of the architecture and training of a voice activity detection automatic speech recognition model (ASR) 700 according to various embodiments of this disclosure. According to certain embodiments, ASR model 700 may be implemented on any suitable processing platform, including, without limitation, device 100 in FIG. 1 or server 200 in FIG. 2. Additionally, ASR 700 may be part (for example, ASR 360 in FIG. 3) of a multi-stage architecture for EEG based speech recognition.

As used in this disclosure, “voice activity” encompasses generating a binary output (i.e., a 0 or 1) based on input features 705, indicating whether a received audio signal or combination of audio and EEG signals comprise speech or other backgrounds. As discussed elsewhere in this disclosure, the shortcomings of existing ASR solutions with regard to processing the speech of individuals with aphasia, apraxia, dysarthria or other conditions distorting or degrading speech beyond the capacity of existing machine learning based speech recognition techniques, extend to speech detection itself. Referring to the non-limiting example of FIG. 7, for both training and prediction, the input features 705 of ASR model 700 are a concatenation (for example, the concatenation obtained at block 355 of FIG. 3) of acoustic representations obtained from EEG signals over a sample time and audio inputs obtained from audio signals over the sample time.

Referring to the illustrative example of FIG. 7, input features are provided GRU 710, which depending on embodiments, comprises a plurality (for example, 512 or 256) of hidden units. To avoid overfitting, the output of GRU 710 is, in some embodiments, provided to a dropout regularization function 715, which drops out the outputs of a predetermined number of hidden units of GRU 710. According to certain embodiments, dropout regularization function 715 is configured to implement a dropout regularization of 0.2, though embodiments with greater or less dropout are possible and within the contemplated scope of this disclosure.

As shown in the explanatory example of FIG. 7, the output of dropout regularization function 715 is provided to a first dense layer 720, which, in this illustrative example, is a time distributed dense layer comprising four hidden units, and then tow a second dense layer 725, which in this example, comprises two hidden units, corresponding to the binary (i.e., “speech=1” or “not speech=0”) output of ASR 700. In certain embodiments, the output of second dense layer 725 is provided to a softmax activation function, which assigns probabilities to each value of a binary output set 735. For example, for a given set of input features 705, softmax activation function 730 may output a probability of 0.8 that input features 705 comprise speech (i.e., a value of “1”) and a probability of 0.2 that input features 705 are not speech (i.e., a value of “0”).

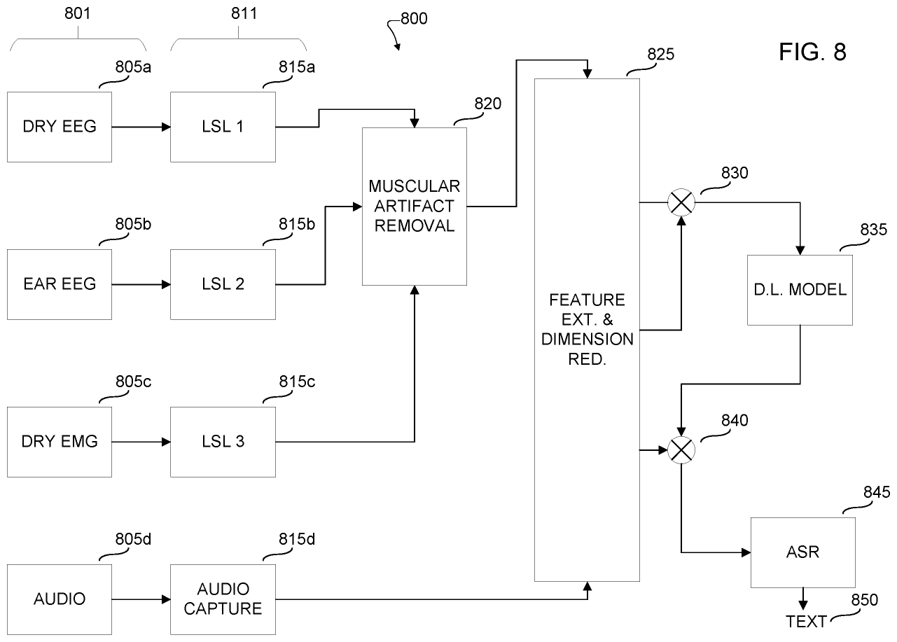

FIG. 8 illustrates an example of an architecture for a real-time speech recognition decoding pipeline 800 according to various embodiments of this disclosure. According to various embodiments, pipeline 800 may be implemented across one or more suitably configured processing platforms (for example, device 100 in FIG. 1 or server 200 in FIG. 2). Additionally, or alternatively, pipeline 800 may be implemented, at least in part, on a cloud or virtualized processing platform (for example, an Amazon AWS container).

Referring to the illustrative example of FIG. 8, decoding pipeline 800 receives, as its inputs, sensor signals 801 from a speaker. According to certain embodiments, sensor signals 801 comprise, at a minimum, a stream of audio signals and EEG signals obtained from the speaker across common time intervals.

In this explanatory example, sensor signals 801 comprise the four sets of sensor signals labeled 805a-805d in FIG. 8. However, embodiments with fewer or greater sensor signals are possible and within the contemplated scope of this disclosure. According to some embodiments, sensor signals 801 include one or more channels of dry EEG sensor signals 805a obtained from a plurality of dry EEG sensors disposed at locations on a speaker's scalp proximate to the regions of the brain responsible for speech function, such as the locations designated Fp1, Fz, F3, F7, FT9, FC5, FT10, FC6, FC 2, F4, F8, Fp2, T7, TP9 and T8 according to the 10-20 EEG sensor placement guidelines. According to some embodiments, dry EEG sensor signals 805a are obtained at a sampling rate of approximately 1000 Hz, and may be amplified, and filtered (for example, to remove 60 Hz noise from nearby mains-powered electrical devices) prior to further processing. While FIG. 8 describes embodiments in which EEG signals are obtained from dry EEG sensors, other embodiments, utilizing wet EEG sensors or combining wet and dry EEG sensors, are possible. According to some embodiments, 30-31 dry EEG sensors may be positioned on points on a speaker's scalp, and the dry EEG sensor signals 805a may comprise 30-31 channels of raw data.

Referring to the illustrative example of FIG. 8, sensor signals 801 comprise one or more channels of ear EEG signals 805b obtained from a plurality of EEG sensors disposed around one or more of a speaker's ears. According to certain embodiments, approximately 9 EEG sensors may be positioned around each of the speaker's ears, and ear EEG signals 805b may, likewise, be sampled at a rate of 1000 Hz.

In certain embodiments, sensor signals 801 further comprise one or more channels of dry EMG sensor signals 805c. Non-invasive EEG sensors (for example, the EEG sensors providing dry EEG signals 805a and ear EEG signals 805b) can detect artifacts from muscle movement in addition to electrical impulses caused by brain activity, it can be desirable to obtain muscle movement sensor data to identify and remove electrical artifacts from muscle, rather than brain, activity. To do this, in some embodiments, one to three dry EMG sensors may be placed along a speaker's chin, and near facial muscle groups whose activity may be detected by EEG sensors. According to various embodiments, to facilitate mapping EMG artifacts to EEG signals, dry EMG sensor signals 805c are obtained at the same sampling rate as dry EEG signals 805a and ear EEG signals 805b. As with dry EEG signals 805a and ear EEG signals 805b, dry EMG signals 805c, may be amplified, filtered and pre-processed before being passed to subsequent stages of pipeline 800.

Referring to the illustrative example of FIG. 8, sensor signals 801 further comprise one or more channels of audio signals 805d from microphone(s) recording audio of the speaker. In some embodiments, for example, where pipeline 800 is implemented on a device carried or worn by a user (for example, a mobile phone or other highly portable processing platform), only a single channel of audio signals may be obtained. In some embodiments, for example, where pipeline 800 is implemented a laboratory or as a permanent installation of a speech therapy center, multiple microphones may be used, and audio signals 805d may comprise a plurality of channels.

According to various embodiments, in pipeline 800, sensor signals 801 are passed to a plurality of stream generation modules 811, which operate in parallel to generate streams of processible data from sensor signals 801. Depending on how sensor signals 801 are collected and pre-processed, stream generation modules 811 may be implemented as hardware (for example, an analog-to-digital converter in conjunction with an audio processor), software or as a combination of hardware and software. As shown in FIG. 8, EEG and EMG signals are converted into data streams, from which the downstream processes of pipeline 800 can, in real-time pull EEG and EMG data in chunks corresponding to a specified temporal resolution (for example, 0.01 seconds) by parallel instances 815a-815c of a lab streaming layer application (“LSL”) (for example, the LSL developed by Kothe et al., and publicly available on Github). According to various embodiments, audio signals 805d are converted to streams of audio data at the specified temporal resolution by one or more audio capture applications (for example, SOUNDTAP or OBS STUDIO).

Referring to the illustrative example of FIG. 8, the stream of dry EEG data output by the first instance of LSL application 815a, the stream of ear EEG data output by the second instance of LSL application 815b, and the stream of dry EMG data output by the third instance of LSL application 815c are passed to block 820, where the dry EMG data is used to identify and remove EMG artifacts from the ear EEG and dry EEG data. In some embodiments, EMG artifacts may be removed using linear regression techniques, such as described with reference to Equation 1 and block 305 of this disclosure.

As shown in the explanatory example of FIG. 8, after muscular artifacts have been removed from the streams of EEG data, the streams of audio data and EEG data are passed to block 825, where EEG features (for example, EEG features such as described with reference to block 305 of FIG. 3, including root mean squared values, kurtosis, and zero crossing rate) are obtained from the EEG data streams, and audio features (for example, MFCC values) are obtained from the audio data. Experimental results have shown that reducing the dimensionality of the EEG feature set can improve overall recognition accuracy for noisy speech. For example, in embodiments where dry EEG signals are obtained from 31 sensors on a speaker's scalp, and ear EEG signals are obtained from nine sensors around each of the speaker's ears, and five features (for example, RMS, Spectral Entropy, Moving Average, Zero-Crossing Rate and Kurtosis) are extracted from each sensor's signal data, then, the EEG data has an initial dimension of 245 (e.g., 31*5+9*5+9*5), which can be computationally expensive to process. According to various embodiments, the dimensionality of the EEG data streams may be reduced using kernel principal component analysis (KPCA), such as with the sklearn.deocmposition.KernelPCA module of SCIKIT. Using a polynomial kernel of degree 3, the EEG data can, in some embodiments, be reduced to a final dimension of degree 10 without any loss of performance.

Referring to the illustrative example of FIG. 8, at block 830, following dimension reduction, the extracted features from the dry EEG data and the extracted features from the ear EEG data are combined and provided as an input to a deep learning model 835 (for example, an instance of deep learning model 303 in FIG. 3), whose inputs comprise EEG features and whose outputs comprise acoustic representations which can be provided as part of a concatenated feature set to one or more ASR models.

As shown in FIG. 8, at block 840, deep learning model 835 outputs acoustic representations which are concatenated with audio data (for example, MFCC's obtained from the audio data stream at block 825) to provide an input feature set for one or more ASR models 845 (for example, the ASR models described with reference to FIGS. 4-7 of this disclosure), which are trained to output one or more recognized text-based outputs 850.

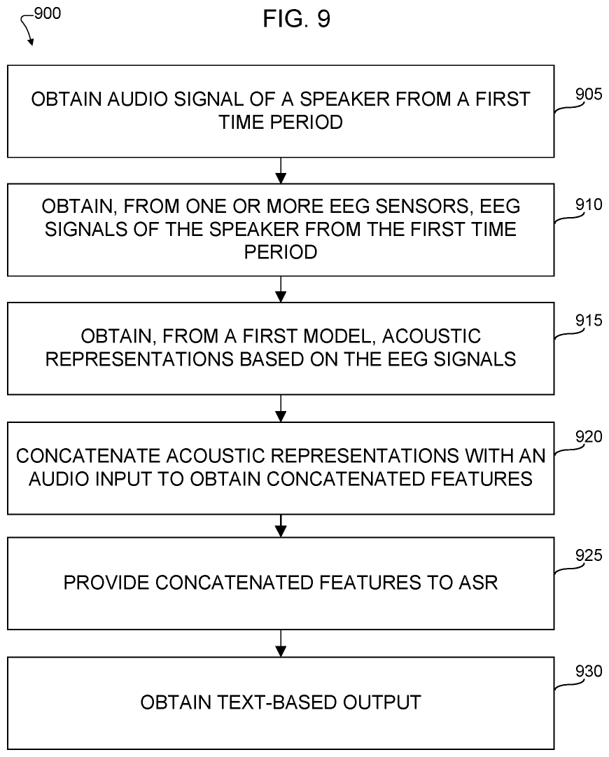

FIG. 9 describes operations of an example method 900 for performing EEG-based speech recognition, according to various embodiments of this disclosure. The operations described with reference to FIG. 9 may be performed on any suitably configured platform, including, without limitation, as an application implemented in part or in whole on electronic device 100 in FIG. 1, server 200 in FIG. 2, or combinations thereof. In some embodiments, the operations of method 900 may be performed in real-time (for example, by implementing a continuous speech recognition model at an electronic apparatus (for example, a smartphone or digital home assistant) configured to operate as a speech prosthetic. According to some embodiments, the operations described with reference to FIG. 9 may comprise part of a control process of a physical system (for example, a voice-controlled wheelchair or internet of things (IoT) system). In certain embodiments, method 900 could be performed at other processor-based apparatus, including, without limitation, an interactive voice response (IVR) system, a voice-controlled appliance, a smart speaker, or a virtual assistant.

Referring to the illustrative example of FIG. 9, at operation 905, the processing platform receives an audio signal of a speaker, obtained by a microphone, from a first time period. According to various embodiments, the obtained audio signal may be an analog electric signal which has been amplified and pre-processed (for example, by being passed through one or more filters). In certain embodiments, the obtained audio signal may, at operation 905, be digitized (for example, through an audio capture application) to create a data stream from a set of audio signals.

As shown in FIG. 9, at operation 910, the processing platform obtains, from one or more EEG sensors (for example, EEG sensors 182 in FIG. 1) EEG signals from the speaker which are measured over the first time period. According to certain embodiments, at operation 910, the obtained EEG signals may be amplified and denoised (for example, using one or more bandpass filters). Further, in some embodiments, EMG artifacts may be removed from the obtained EEG signals, and the EEG signals themselves may be digitized and converted into one or more streams of EEG data, which can be fetched for downstream processing in batches of data corresponding to predetermined time increments.

Referring to the explanatory example of FIG. 9, at operation 915, a set of acoustic representations (for example, the acoustic representations obtained at block 320 in FIG. 3, or from deep learning model 835 in FIG. 8) are obtained by providing the EEG signals (or features obtained therefrom, such as described with reference to block 825 of FIG. 8) to a pretrained deep learning model (for example, model 303 in FIG. 3) to obtain a set of acoustic representations. According to various embodiments, the acoustic representations obtained at operation 915, while obtained from EEG signals, are of a type (for example, Mel Frequency Cepstral Coefficients) which maps to the input feature space of one or more speech recognition models.

According to various embodiments, at operation 920, the acoustic representations obtained at operation 920 are concatenated (for example, as described with reference to block 355 of FIG. 3) with an audio input (for example, MFCCs obtained from the audio signal obtained at operation 905) to produce a set of concatenated features, wherein the set of concatenated features is provided, at operation 920 to one or more ML automatic speech recognition models (for example, one or more models as described with reference to the examples of FIGS. 4-7). As shown in the explanatory example of FIG. 9, the ASR provides, based on the concatenated features, a text-based output, wherein the text-based output is based on the ASR's recognition of one or more textual aspects (for example, recognition of a sentence, recognition of a spoken word, identification of a speaker, identification of whether an audio signal is associated with human speech).

The embodiments described with reference to FIGS. 1-9 are intended to illustrate, rather than limit the scope of this disclosure, and skilled artisans will appreciate that further embodiments and variations of the structures and principles set forth herein are possible and within the contemplated scope of this disclosure.

What is claimed is:

1. A method of electroencephalography (EEG) based speech recognition, comprising:
obtaining, from a microphone, an audio signal of a speaker from a first time period;
obtaining, from one or more EEG sensors, EEG signals of the speaker from the first time period;
obtaining, from a first model, acoustic representations based on the EEG signals;
concatenating the obtained acoustic representations with an audio input based on the audio signal to obtain concatenated features;
providing the concatenated features to an automatic speech recognition model (ASR); and
obtaining, from the ASR model, a text-based output.

2. The method of claim 1, wherein the ASR model is at least one of an isolated speech recognition model, a continuous speech recognition model, a speaker identification model or a voice activity detection model.

3. The method of claim 1, wherein the one or more EEG sensors comprise at least one of a non-invasive wet EEG sensor, a non-invasive dry EEG sensor, or an ear EEG sensor.

4. The method of claim 1,
wherein obtaining EEG signals comprises obtaining first EEG signals from a dry EEG sensor, and second EEG signals from an ear EEG sensor, and further comprising:
obtaining, from an electromyography (EMG) sensor, EMG signals of the speaker from the first time period;
filtering EMG artifacts from the first EEG signals and the second EEG signals based on the EMG signals;
reducing a dimensionality of the first EEG signals;
reducing a dimensionality of the second EEG signals; and
concatenating the first and second EEG signals,
wherein providing the EEG signals to a first model to obtain acoustic representations comprises providing the concatenated first and second EEG signals to the first model.

5. The method of claim 4,
wherein reducing the dimensionality of the first EEG signals comprises performing a first kernel principal component analysis (KPCA) to reduce the dimensionality of the first EEG signals.

6. The method of claim 1, wherein the audio input comprises Mel frequency cepstral coefficients (MFCC) extracted from the audio signal.

7. The method of claim 1, wherein the first model comprises:
a regression model comprising a gated regression unit (GRU) with a first plurality of hidden units; and
a time distributed dense layer comprising a second plurality of hidden units and a linear activation function.

8. The method of claim 1, wherein the automatic speech recognition model is an isolated speech recognition model comprising:
a GRU with a plurality of hidden units;
a dropout regularization function applied to the GRU;
a dense layer; and
a softmax activation function,
wherein the softmax activation function outputs label prediction probabilities.

9. The method of claim 1, wherein the ASR model is a continuous speech recognition model comprising:
a GRU with a plurality of hidden units;
a dense layer;
a softmax activation function; and
a connectionist temporal classification (CTC) loss function.

10. The method of claim 1, wherein obtaining the acoustic representations comprises:
extracting EEG features from the EEG signal; and
providing the EEG features to the first model to obtain the acoustic representations,
wherein the EEG features comprise at least one of a root mean square, a zero-crossing rate, a moving window average, a kurtosis value and a power spectral entropy value.

11. An apparatus for performing electroencephalography (EEG) based speech recognition, comprising:
an input/output interface; and
a processor configured to:
obtain, from a microphone, via the input/output interface, an audio signal of a speaker from a first time period,
obtain, from one or more EEG sensors, via the input/output interface, EEG signals of the speaker from the first time period,
obtain, from a first model, acoustic representations based on the EEG signals,
concatenate the obtained acoustic representations with an audio input based on the audio signal to obtain concatenated features,
provide the concatenated features to an automatic speech recognition model (ASR), and
obtain, from the ASR model, a text-based output.

12. The apparatus of claim 11, wherein the ASR model is at least one of an isolated speech recognition model, a continuous speech recognition model, a speaker identification model or a voice activity detection model.

13. The apparatus of claim 11, wherein the one or more EEG sensors comprise at least one of a non-invasive wet EEG sensor, a non-invasive dry EEG sensor, or an ear EEG sensor.

14. The apparatus of claim 11,
wherein obtaining EEG signals comprises obtaining first EEG signals from a dry EEG sensor, and second EEG signals from an ear EEG sensor, and wherein the processor is further configured to:
obtain, from an electromyography (EMG) sensor, via the input/output interface, EMG signals of the speaker from the first time period,
filter EMG artifacts from the first EEG signals and the second EEG signals based on the EMG signals,
reduce a dimensionality of the first EEG signals,
reduce a dimensionality of the second EEG signals, and
concatenate the first and second EEG signals, and
provide the concatenated first and second EEG signals to the first model.

15. The apparatus of claim 14,
wherein reducing the dimensionality of the first EEG signals comprises performing a first kernel principal component analysis (KPCA) to reduce the dimensionality of the first EEG signals.

16. The apparatus of claim 11, wherein the audio input comprises Mel frequency cepstral coefficients (MFCC) extracted from the audio signal.

17. The apparatus of claim 11, wherein the first model comprises:
a regression model comprising a gated regression unit (GRU) with a first plurality of hidden units; and
a time distributed dense layer comprising a second plurality of hidden units and a linear activation function.

18. The apparatus of claim 11, wherein the automatic speech recognition model is an isolated speech recognition model comprising:
a GRU with a plurality of hidden units;
a dropout regularization function applied to the GRU;
a dense layer; and
a softmax activation function,
wherein the softmax activation function outputs label prediction probabilities.

19. The apparatus of claim 11, wherein the ASR model is a continuous speech recognition model comprising:
a GRU with a plurality of hidden units;
a dense layer;
a softmax activation function; and
a connectionist temporal classification (CTC) loss function.

20. The apparatus of claim 11, wherein obtaining the acoustic representations comprises:
extracting EEG features from the EEG signal; and
providing the EEG features to the first model to obtain the acoustic representations,
wherein the EEG features comprise at least one of a root mean square, a zero-crossing rate, a moving window average, a kurtosis value and a power spectral entropy value.
